SEQUENCE LISTING (2 sequences)

SEQ ID NO: 1 - protein, 7 aa, synthetic construct
  REGION 1..7: note = peptyd 1
KIINLKR

SEQ ID NO: 2 - protein, 8 aa, synthetic construct
  REGION 1..8: note = peptyd 2
NYMALRRY

(19) United States
(12) Patent Application Publication
(10) Pub. No.: US 2026/0235624 A1
(43) Pub. Date: Aug. 13, 2026

(54) PEPTIDES AND THEIR LABELLED DERIVATIVES SUITABLE FOR USE IN THE DIAGNOSIS OF DIABETES OR ITS COMPLICATIONS

(71) Applicant: Instytut Medycyny Doswiadczalnej i Klinicznej im. Miroslawa Mossakowskiego Polskiej Akademii Nauk, Warszawa (PL)

(72) Inventors: Agnieszka PIWKOWSKA, Gdansk (PL); Dorota ROGACKA, Gdansk (PL); Adam LESNER, Pepowo (PL); Magdalena WYSOCKA, Gdansk (PL); Natalia GRUBA, Keblowo (PL); Irena AUDZEYENKA, Gdañsk (PL)

(73) Assignee: Instytut Medycyny Doswiadczalnej i Klinicznej im. Miroslawa Mossakowskiego Polskiej Akademii Nauk, Warszawa (PL)

(21) Appl. No.: 19/156,437

(22) PCT Filed: Feb. 14, 2024
(86) PCT No.: PCT/PL2024/050012

§ 371 Date: Aug. 14, 2025

(30) Foreign Application Priority Data
Feb. 14, 2023 (PL) P.443779

(51) Int. Cl. G01N 33/68; C07K 7/06; G01N 33/58

(52) U.S. Cl. CPC G01N 33/6893; C07K 7/06; G01N 33/582; G01N 2333/96486; G01N 2410/00; G01N 2800/042; G01N 2800/347

(57) ABSTRACT

Novel peptide substrates hydrolysed by ADAM10 or ADAM17 are disclosed, as well as their applications in the diagnosis of diabetes or its complications.

The invention relates to novel peptide substrates hydrolysed by the proteolytic enzyme ADAM10 or ADAM17 and their use in the diagnosis of diabetes or its complications.

One of the adverse effects of diabetes is damage to kidney function (so-called diabetic nephropathy), resulting in the pathologic presence of proteins in the patient's urine.

The diabetic state is evidenced by increased activity of the proteolytic enzymes ADAM10/ADAM17, observed even before sugar is present in the urine (Curr. Issues Mol. Biol. 2022, 44, 4517-4527).

Currently, a common method for diagnosing diabetes is to measure plasma glucose levels. This method is invasive and significantly susceptible to the influence of several external factors, such as exercise or food intake prior to blood collection.

There is still a need to provide a method for diagnosing diabetes, especially its earlier stages, that could be easily performed and would not involve collecting and analysing the patient's blood.

Unexpectedly, the above problem has been solved in the present invention.

### Essence of the Invention

The subject of the invention is a peptide comprising an amino acid sequence selected from KIINLKR (SEQ ID NO: 1) or NYMALRRY (SEQ ID NO: 2) or a labelled derivative thereof.

Preferably, it has an amino acid sequence selected from SEQ ID NO: 1-2.

Preferably, the labelled derivative of the peptide of the invention is a fluorescently labelled derivative, preferably a compound of formula 1 or 2:

(formula 1)
ABZ-Lys-Ile-Ile-Asn-Leu-Lys-Arg-Tyr(3-NO₂)-NH₂
(formula 2)
ABZ-Asn-Tyr-Met-Ala-Leu-Arg-Arg-Tyr(3-NO₂)-NH₂.

A further subject of the invention is the peptide of the invention or derivative thereof as defined above for use in diagnosis, in particular of diabetes or its complications.

Preferably, the diagnosed complication of diabetes is diabetic nephropathy.

Another subject of the invention is a method for diagnosing diabetes or its complications characterised in that it comprises the following steps:
a) in-vitro contacting a urine sample from a patient with a substrate hydrolysed by the proteolytic enzyme ADAM10 or ADAM17,
b) testing for the presence of a product of such hydrolysis, whereby the detection of such hydrolysis product indicates the detection of diabetes mellitus or its complications in the patient under investigation,
wherein the peptide of the invention or derivative thereof as defined above is used as the substrate hydrolysed by the proteolytic enzyme ADAM10 or ADAM17.

Preferably, the diagnosed complication of diabetes is diabetic nephropathy.

Preferably, in step a) the concentration of the substrate hydrolysed by the proteolytic enzyme ADAM10 or ADAM17 in the sample to be analysed is at least 4.578×10⁻¹⁰ M (for ADAM10); 1.87×10⁻⁵ M (for ADAM17).

Preferably, in step a) the substrate hydrolysed by the proteolytic enzyme ADAM10 or ADAM17 is additionally contacted with a measurement buffer containing 25 mM Tris-Cl, 2.5 μM ZnCl₂ and 0.005% Brij-35, preferably at pH 9.0.

Preferably, a fluorescently labelled substrate hydrolysed by the proteolytic enzyme ADAM10 or ADAM17 is used, and in step b) the increase in fluorescence of the of the test sample is measured.

### DETAILED DESCRIPTION OF THE INVENTION

The essence of the invention is a set of two peptides (substrates), preferably with fluorogenic properties. These peptides are hydrolysed in the presence of proteolytic enzymes (ADAM10 or ADAM 17) released and present in the urine of diabetic patients.

The proposed application for the new compounds is the diagnosis of diabetes and associated diabetic nephropathy. The diagnostic potential of the method, based on the assessment of the hydrolytic activity of proteolytic enzymes in human urine by measuring the fluorogenic activity of their substrates, has been confirmed by the results obtained by the inventors. It was observed that the activity of proteolytic enzymes in the urine of diabetic patients was significantly higher than in people with normal blood glucose levels. The results also showed that the new fluorogenic peptide substrates could serve directly as easy-to-use diagnostic markers of proteolytic enzyme activity, and indirectly, of diabetes.

When using labelled (e.g. fluorescently) derivatives of these peptides, their hydrolysis can be easily detected by observing an increase in the concentration of the labelled degradation products in the test sample (e.g. an increase in fluorescence).

On the other hand, in the urine samples of healthy subjects, which should not contain the enzymes ADAM10 or ADAM 17, also the hydrolysis products of the peptides of the invention, and thus also e.g. fluorescence of the products of degradation of their fluorescently labelled derivatives, should not be present.

In an preferred embodiment of the invention, involving the use of fluorescently labelled derivatives of the peptides of the invention, the measurement of the fluorogenic activity of the new compounds in urine is very rapid (a few minutes) and requires a test sample of small volume (about 0.5 mL).

In order to better explain the essence of the invention, the description of the invention is supplemented by the embodiments given below and illustrated by the accompanying figures, whereby:

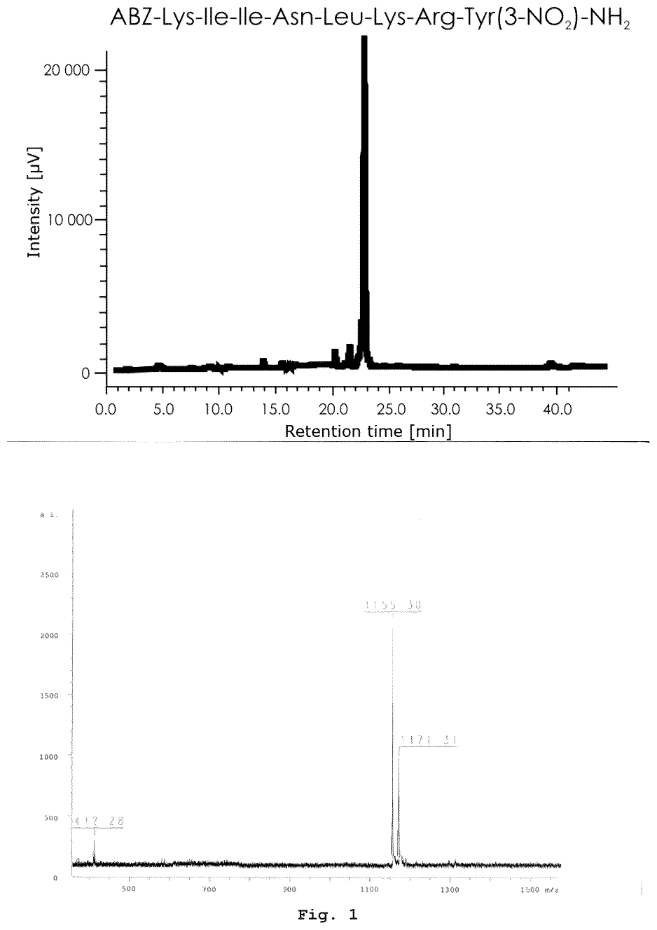

FIG. 1 shows the physicochemical characterisation of an exemplary compound for the detection of ADAM-10 protease, while

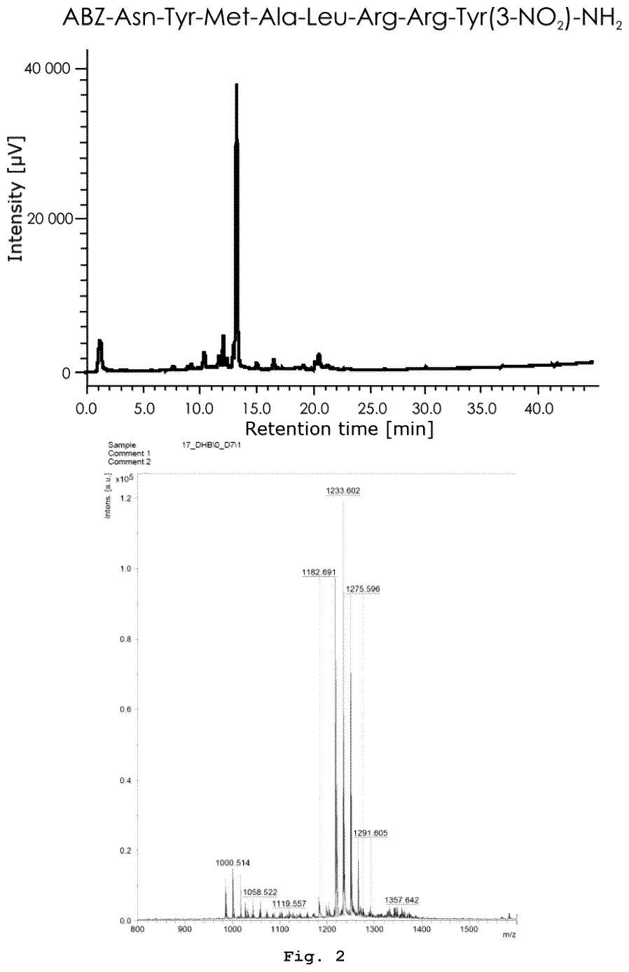

FIG. 2 shows the physicochemical characterisation of an exemplary compound for the detection of ADAM-17 protease, and

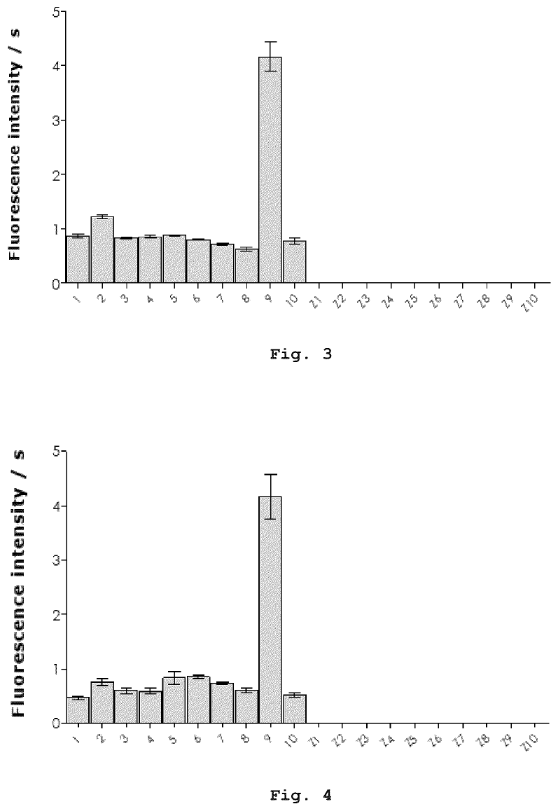

FIGS. 3 and 4 show the results of the measurements described in Example 3.

### EXAMPLE 1. PEPTIDES OF THE INVENTION AND THEIR LABELLED DERIVATIVES

Peptides of the invention have the following amino acid sequences: KIINLKR (SEQ ID NO: 1) or NYMALRRY (SEQ ID NO: 2).

These peptides can be obtained by any known synthesis methods.

Examples of such methods include solid-phase synthesis using Fmoc/tBu chemistry. It is now a widely available and widely used technique based on the method proposed in 1963 by R. B. Merrifield (Solid Phase Peptide Synthesis. I. The Synthesis of a Tetrapeptide, R. B. Merrifield, J. Am. Chem. Soc. 1963, 85, 14, 2149-2154, https://doi.org/10.1021/ja00897aC25).

Using the method described above, peptides with sequences 1 and 2 were obtained. Their purity and structure were confirmed by RP HPLC and MS methods obtaining the following results, see FIGS. 1 and 2.

In preferred embodiments of the invention, labelled derivatives of these peptides are used. This allows easier detection of their hydrolysis products.

It is possible to use peptides labelled by any known method. Such methods may in particular be the measurement of fluorescence over time.

For reasons of ease and safety of use, fluorescently labelled peptides are used in an preferred embodiment of the invention. These can be obtained by any known labelling method involving the introduction of a known fluorescent marker into the peptide structure. Examples of such fluorescent group donors are ortho-aminobenzoic acid (ABZ), 7-methoxycoumarin-4-acetic acid (MCA) or 5-(dimethylamino)naphthalene-1-sulfonyl chloride (DANSYL), allowing easy attachment of the fluorescent label to the N-terminal amino group of the peptide. Methods for introducing their structure are well known to the skilled in the art and have been exemplified in the following publications:
1. Lȩgowska M, Wysocka M, Burster T, Pikula M, Rolka K, Lesner A. Ultrasensitive internally quenched substrates of human cathepsin L. Anal Biochem. 2014 Dec. 1; 466:30-7.
2. Wysocka M, Lesner A, Gruba N, Korkmaz B, Gauthier F, Kitamatsu M, Lȩowska A, Rolka K. Three wavelength substrate system of neutrophil serine proteinases. Anal Chem. 2012 Aug. 21; 84(16):7241-8.
3. Lȩowska M, Hamon Y, Wojtysiak A, Grzywa R, Sieńczyk M, Burster T, Korkmaz B, Lesner A. Development of the first internally-quenched fluorescent substrates of human cathepsin C: The application in the enzyme detection in biological samples. Arch Biochem Biophys. 2016 Dec. 15; 612:91-102.

Examples of fluorescently labelled peptides of the invention are the following compounds:

ABZ-Lys-Ile-Ile-Asn-Leu-Lys-Arg-Tyr(3-NO₂)—NH₂ (derivative 1),

ABZ-Asn-Tyr-Met-Ala-Leu-Arg-Arg-Tyr(3-NO₂)—NH₂ (derivative 2).

### EXAMPLE 2. PREPARATION OF FLUORESCENTLY LABELLED DERIVATIVES OF THE PEPTIDES OF THE INVENTION

Fluorescently labelled derivatives of the peptides of the invention can be obtained by any known means. For example, they can be obtained using a method for the preparation of chromogenic peptides based on a solid phase process using a resin with Fmoc group, which is removed in the course of the reaction. For example, this could be an amide resin, such as TentaGel S RAM or RinkAmide, but any other commercially available resin could also be used. The resin used to carry out this process should be properly prepared. The preparation of this resin involves increasing its volume by repeated washing with hydrophobic solvents. Preferably, a resin with a substitution of 0.23 mmol/g is used. The Fmoc protecting group should be removed from this resin by washing with a 20% solvent solution.

Subsequently, the known processes for the preparation of chromogenic peptides involve the attachment of the individual components under appropriate time and stoichiometric conditions.

This attachment process consists of successive steps in which the individual components (amino acid derivatives) are attached, the residues are washed away, and the N-amino protecting group is sequentially removed and washed again. This cycle is repeated for each amino acid residue. The resulting peptide is cleaved off from the resin by reaction under acidic conditions. The solution is then separated from the resin by filtration, and the peptide is then precipitated from the resulting solution with a non-polar solvent. The peptide precipitate thus obtained is centrifuged.

The method for the synthesis of compounds of the invention is based on a solid phase process using a resin, preferably with Fmoc group, the resin being prepared prior to the process by increasing its volume (swelling) by repeated washing with hydrophobic solvents, preferably dimethylformamide, methylene chloride or N-methylpyrrolidone, and removing the Fmoc protecting group, preferably by washing with a 10-30% piperidine solution, in solvents such as dimethylformamide, methylene chloride or N-methylpyrrolidone.

The method is then carried out in the following steps:
a) attachment of Fmoc-Tyr(NO₂),
b) attachment of further amino acid residues to Fmoc-Tyr(NO₂) is carried out by reaction with an amino acid derivative, washing with DMF and MeOH and gently drying, after which the resulting intermediate compound is acylated by successive attachment of the fragment: ABZ-Lys-Ile-Ile-Asn-Leu-Lys-Arg;
c) removal of the Fmoc protecting group is carried out by washing with a solution of 10-30% piperidine in DMF and subsequent washes with each of the solvents: DMF, isopropanol and methylene chloride,
d) cleavage of the peptide from the resin is carried out using a TFA:phenol:water:TIPS mixture at ratios of 88:5:5:2 v/v/v/v respectively, the mixture being stirred for at least one hour, preferably three hours, and the precipitate obtained is filtered off under reduced pressure, followed by washing with diethyl ether and the peptide obtained is centrifuged;
e) the preparation of the finished product is carried out by ultrasound-assisted dissolving of the peptide in water, followed by freeze-drying.

Using the method described above, derivatives 1 and 2 were obtained. Their purity and structure were confirmed by RP HPLC and MS, obtaining the results shown in FIGS. 1 and 2.

### EXAMPLE 3. METHOD OF THE INVENTION FOR DIAGNOSING DIABETES MELLITUS

20 μL of a 45 μM substrate solution of the peptide of the invention or its labelled derivative were mixed with 100 μL of the measurement buffer (25 mM Tris-Cl, 2.5 μM ZnCl₂ and 0.005% Brij-35, pH 9.0) and 80 μL of test urine from a patient diagnosed with diabetes or a healthy person (control).

When fluorescently labelled derivatives were used, measurements were carried out in triplicate in a 96-well plate using a CLARIOStar plate reader (BMG Labtech, Ortenberg, Germany) at 37° C. for 30 minutes. After this time, the measurement was finished and the increase in fluorescence was determined. If a fluorescence increase of more than 0.0001 U/sec was obtained, the urine sample tested was considered to originate from a person with diabetes.

The results obtained are shown in the tables below.

TABLE 1
Increase in concentration of labelled degradation
products in the urine sample obtained for solutions containing
the substrate of the invention hydrolysed by ADAM17. Samples
1-10 were urine samples obtained from diabetic patients, samples
Z1-Z10 were control urine samples (healthy subjects).

| | | Standard | |
|---|---|---|---|
| Sample | Mean | deviation | SEM |
| 1 | 0.810000 | 0.92000 | 0.8800 |
| 2 | 1.150000 | 1.31000 | 1.2100 |
| 3 | 0.810000 | 0.83000 | 0.8500 |
| 4 | 0.820000 | 0.90000 | 0.8500 |
| 5 | 0.900000 | 0.87000 | 0.8800 |
| 6 | 0.810000 | 0.79000 | 0.8000 |
| 7 | 0.680000 | 0.75000 | 0.7200 |
| 8 | 0.570000 | 0.69000 | 0.6200 |
| 9 | 3.660000 | 4.59000 | 4.2300 |
| 10 | 0.700000 | 0.87000 | 0.7500 |
| Z1 | 0.000000 | 0.00000 | 0.0000 |
| Z2 | 0.000000 | 0.00000 | 0.0000 |
| Z3 | 0.000000 | 0.00000 | 0.0000 |
| Z4 | 0.000000 | 0.00000 | 0.0000 |
| Z5 | 0.000000 | 0.00000 | 0.0000 |
| Z6 | 0.000000 | 0.00000 | 0.0000 |
| Z7 | 0.000000 | 0.00000 | 0.0000 |
| Z8 | 0.000000 | 0.00000 | 0.0000 |
| Z9 | 0.000000 | 0.00000 | 0.0000 |
| Z10 | 0.000000 | 0.00000 | 0.0000 |

TABLE 2
Increase in concentration of labelled degradation
products in the urine sample obtained for solutions containing
the substrate of the invention hydrolysed by ADAM10. Samples
1-10 were urine samples obtained from diabetic patients, samples
Z1-Z10 were control urine samples (healthy subjects).

| | | Standard | |
|---|---|---|---|
| Sample | Mean | deviation | SEM |
| 1 | 0.530000 | 0.42000 | 0.4500 |
| 2 | 0.880000 | 0.65000 | 0.7200 |
| 3 | 0.700000 | 0.50000 | 0.5900 |
| 4 | 0.690000 | 0.50000 | 0.5800 |
| 5 | 1.020000 | 0.62000 | 0.8500 |
| 6 | 0.810000 | 0.83000 | 0.9200 |
| 7 | 0.720000 | 0.77000 | 0.7200 |
| 8 | 0.550000 | 0.55000 | 0.6900 |
| 9 | 4.000000 | 3.54000 | 4.9400 |
| 10 | 0.490000 | 0.45000 | 0.6000 |
| Z1 | 0.000000 | 0.00000 | 0.0000 |
| Z2 | 0.000000 | 0.00000 | 0.0000 |
| Z3 | 0.000000 | 0.00000 | 0.0000 |
| Z4 | 0.000000 | 0.00000 | 0.0000 |
| Z5 | 0.000000 | 0.00000 | 0.0000 |
| Z6 | 0.000000 | 0.00000 | 0.0000 |
| Z7 | 0.000000 | 0.00000 | 0.0000 |
| Z8 | 0.000000 | 0.00000 | 0.0000 |
| Z9 | 0.000000 | 0.00000 | 0.0000 |
| Z10 | 0.000000 | 0.00000 | 0.0000 |

The results presented in Tables 1 and 2 are also shown in FIGS. 3 and 4, respectively.

Conclusions: The results obtained confirm that only urine samples from diabetic subjects exhibited proteolytic activity resulting in hydrolysis of the peptides of the invention or their derivatives. The determination of the presence of hydrolysis products was particularly easy when fluorescently labelled derivatives were used.

1. A peptide containing an amino acid sequence selected from sequence nos. 1-2 or a labelled derivative thereof.

2. The peptide of claim 1, wherein it has an amino acid sequence selected from SEQ ID NO: 1-2.

3. A labelled derivative of the peptide of claim 1, wherein it is a fluorescently labelled derivative, preferably a compound of formula 1 or 2:
ABZ-Lys-Ile-Ile-Asn-Leu-Lys-Arg-Tyr(3-NO₂)—NH₂ (formula 1)
ABZ-Asn-Tyr-Met-Ala-Leu-Arg-Arg-Tyr(3-NO₂)—NH₂ (formula 2).

4. The peptide or derivative thereof as defined in claim 1 for use in the diagnosis, particularly of diabetes or its complications.

5. A peptide or derivative thereof for the use of claim 4, wherein the diagnosed complication of diabetes is diabetic nephropathy.

6. A method for diagnosing diabetes or its complications, wherein it comprises the following steps:
a) in-vitro contacting of a urine sample from a patient with a substrate hydrolysed by the proteolytic enzyme ADAM10 or ADAM17,
b) testing for the presence of a product of such hydrolysis, whereby the detection of such hydrolysis product indicates the detection of diabetes mellitus or its complications in the patient under investigation,
wherein the substrate hydrolysed by the proteolytic enzyme ADAM10 or ADAM17 is the peptide or derivative thereof as defined in claim 1.

7. The method of claim 6, wherein the diagnosed complication of diabetes is diabetic nephropathy.

8. The method of claim 6, wherein in step a) the concentration of the substrate hydrolysed by the proteolytic enzyme ADAM10 or ADAM17 in the sample to be analysed is at least 4.578×10⁻¹⁰ M (for ADAM10); 1.87×10⁻⁵ M (for ADAM17).

9. The method of claim 6, wherein in step a) the substrate hydrolysed by the proteolytic enzyme ADAM10 or ADAM17 is additionally contacted with a measurement buffer containing 25 mM Tris-Cl, 2.5 μM ZnCl₂ and 0.005% Brij-35, preferably at pH 9.0.

10. The method of claim 6, wherein a fluorescently labelled substrate hydrolysed by the proteolytic enzyme ADAM10 or ADAM17 is used, and in step b) the increase in fluorescence of the test sample is measured.
